(19) United States
(12) Patent Application Publication
(10) Pub. No.: US 2015/0212057 A1
(43) Pub. Date: Jul. 30, 2015

(54) Wearable Air Quality Monitor

(71) Applicant: Peter Darveau, Oakville (CA)

(72) Inventor: Peter Darveau, Oakville (CA)

(21) Appl. No.: 14/162,897

(22) Filed: Jan. 24, 2014

(51) Int. Cl. G01N 33/00

(52) U.S. Cl. CPC G01N 33/004; G01N 33/0062

(57) ABSTRACT

A wearable digital air quality monitor, which can display the exact sampling of carbon monoxide (CO) and particulate matter (PM) data, and a program capable of performing the functions of sampling, interpreting the sampled data through an interpolation function on the sensors' response curve, controlling the heating element to the exact specifications of the metal oxide sensor, and calibration. Exposed to clean air environment, the CPU controls the heating element cycle to ensure a optimum sensor sensitivity to the CO and it then calibrates the sensor. In subsequent steps, the CPU samples and averages CO data taken at a predefined 50 millisecond interval and PM_{2.5} at a predefined 3 second interval, then determines the amount of carbon monoxide by performing an inverse logarithmic function that follows the sensor's response curve. For PM_{2.5} measurements, the data is normalized to determine a total particle count per centimeter cubed (cm³) and then applies a multiplier determining the PM_{2.5} density. This multiplier is based on air pollution studies conducted in North America and Europe. The CO and PM_{2.5} measurements are displayed on a dual-line BCD display. The device also has communication interfacing capabilities providing an external link to other computers, hand-held devices such as smartphones and tablets.

### RELATED U.S APPLICATION DATA

Application No. U.S. 61/769,121 filed Feb. 25, 2013 by Peter Darveau

### REFERENCES

U.S. Pat. No. 4,355,533

U.S. Pat. No. 4,229,968

U.S. Pat. No. 3,732,411

U.S. Pat. No. 3,691,364

U.S. Pat. No. 8,220,310

U.S. Pat. No. 8,178,047

U.S. Pat. No. 7,493,795

U.S. Pat. No. 7,395,175

U.S. Pat. No. 4,787,239

U.S. Pat. No. 4,414,839

U.S. Pat. No. 7,104,113

U.S. Pat. No. 8,176,768

U.S. Pat. No. 8,166,800

U.S. Pat. No. 6,930,586

U.S. Pat. No. 6,606,897

U.S. Pat. No. 6,234,006

U.S. Pat. No. 5,356,594

U.S. Pat. No. 4,189,725

U.S. Pat. No. 8,186,234

U.S. Pat. No. 7,111,496

U.S. Pat. No. 4,792,433

U.S. Pat. No. 4,586,143

### BACKGROUND OF THE INVENTION

1. Field of the Invention

This invention relates to a wearable digital air quality monitor, which can display the exact sampling of carbon monoxide (CO) and particulate matter (PM_{2.5}) data, and a program capable of performing the functions of sampling, interpreting the sampled data through an interpolation function on the sensor s'response curve, controlling the heating element for the CO metal oxide sensor and pulsing the PM sensor to the exact specifications as required, and calibration.

2. Problems to be solved by the Invention.

Air quality monitors require the sampling of gas and a reference gas for calibration purposes and often require chambers, each one having a sensor for isolating the gases for analysis. Calibrating the device is not an automatic function and often requires a certain amount of technical expertise. When heating of the sensor is required, power consumption is high making it difficult to have a long-lasting battery-operated monitor. These devices, which often do Gas or Particulate Matter rather than both together, aren't trendy-looking but are bulky which are strong deterrents for acceptance by the general public. The prior art doesn't speak to a small, light-weight device that can be worn and carried like a cell phone and that is made up of sensors that give a consumer the ability to read the levels of particulate matter and CO in a given location and at a given time. Also, communication interfaces for transferring data or interfacing with other computers or smart devices isn't available. Accordingly, there is a need to improve the integration of all these functions into one small wearable unit using the latest sensing and circuit technologies and combine them into a single device capable of monitoring and displaying the two most common airborne pollutants affecting public respiratory health globally.

### BRIEF DESCRIPTION OF THE INVENTION

The present invention has been developed to meet a need in the marketplace and solve the above-described problems, and an object thereof is to provide a wearable air quality monitor that integrates the functions of sampling the target gas (CO) and Particulate Matter (PM), executing both the heating cycle for the CO sensor and the timing pulsing of the PM sensor to accurately measure the specific gas and airborne particles being sampled, the calibration and associated gas and particle sampling routines associated with them, a display of the measurements of specific gas and PM_{2.5} concentrations and provide for a external communication interface, such as USB, to integrate and transfer data to other consumer devices.

The wearable air quality monitor is versatile and meets the needs of a wide range of populations in the developed and developing world. The device is designed as a ultra-slim and light-weight wearable device making it possible to strap it on the arm or insert in a pocket. The monitor includes an internal processor for processing the sampling of the ambient air, calibration, controlling the heating and pulsing cycles required by the sensors, displaying the data on the two-line display. The built-in communications are readily available to connect to another computer or smart device.

A specific embodiment of the invention provides a sensing apparatus that includes a housing and the pre-wired and pre-connected sensors. The monitoring occurs immediately when the monitor is powered. The monitor is configured to automatically start calibration, identify and quantify, based on the sampling routine executed by the processor, the clean air and sensor resistance and voltage outputs of the sensors. The processor executes the appropriate heating cycle for the CO sensor and the pulsing of the PM sensor ensuring the accuracy of the measurements.

According to a first aspect, the present invention is made up of a carbon monoxide sensor and a particulate matter sensor. The carbon monoxide detector is a solid-state sensor detecting the concentration of CO by measuring its conductivity which changes with the interaction with the concentration of the gas molecules. The detection is accomplished using a load resistor connected in series with the sensor creating a voltage divider circuit. Voltage measurements, proportional to gas concentrations, are taken across the load resistor whose value is chosen for maximum sensitivity. These connections are inputs to the micro-controller.

A heating element built-into the CO sensor is used to regulate the sensor temperature to match the response of the sensor to carbon monoxide ensuring the accuracy of the measurements taken. The terminals of the heater element are regulated and controlled by the micro-controller and the internal voltage regulator. For optimal performance, the circuit provides a 5.0 VDC voltage for 60 seconds and 1.5 VDC for 30 seconds. Measurements across the load resistor are taken during the low voltage (1.5 VDC) part of the heating cycle for optimal accuracy.

In a second aspect of the present invention, the particulate matter is measured by an laser diode optical sensor. An infrared emitting diode and a phototransistor are diagonally arranged within the sensor measuring light intensity providing a sampling based on light scattering theory. It detects the reflected light according to the concentration of particulate matter (PM) of 0.5 microns and up in the air. The PM is detected by measuring the voltage present on the output of the sensor which changes proportionately with the concentration.

In the air quality detection monitor, the concentration of carbon monoxide and particulate matter is determined by measuring voltage across the terminals of the sensors. Since the sensors need only clean air as a reference for accurate measurements, calibration is first required. Once the monitor is powered, the program executed by the micro-controller goes through a series of steps for calibrating, sampling, calculating and displaying the concentrations. The program first starts a heating cycle bringing the sensor to the optimal operating temperature for carbon monoxide detection. Once the operating temperature is reached, the calibration starts whereby the clean air resistance of the detector is measured and calculated after the number of calibration samples has been reached. The program then samples the detector resistance by measuring the voltage across the load resister at a predefined number of samples and a sampling rate. For every cycle, the carbon monoxide concentration is calculated by interpolating the ratio of the detector resistance and the clean air resistance on the sensitivity chart and the concentration of the gas. It is then displayed on the first line of the display screen.

According to the second aspect of the invention, the PM detector does not use the variable conductivity of a metal oxide but rather light deflection. For this aspect, calibration is not required. Therefore, the measured PM is a direct relationship between the terminal voltage of the detector and the concentration of particulate matter. The sensitivity of the sensor does however require an average to be continuously calculated over a range of 100 samples kept in an array in the program of the micro-controller. This method reduces the amount of bounce displayed on the display. For every program cycle, the average of the particulate matter is calculated and then displayed on the second line of the display screen.

Therefore, since all the necessary configuration functions are performed on the detectors as part of a comprehensive program executed by the micro-controller, the measurements can be displayed accurately for air quality monitoring purposes.

A third aspect of the invention includes a communication interface allowing the wearable air quality monitor to communicate to another computer.

The present invention has a communication interface with receive (Rx) and transmit (Tx) signals.

In a fourth aspect of the invention, all the circuitry and the display are contained in a single housing that is light-weight and wearable. There are 2 connections possible in the housing allowing the input signals of carbon monoxide and PM sensors. The detectors are pre-wired and connected allowing a simple step to use the monitor without the need for any inlets for gas samples or the use of any tools.

### BRIEF DESCRIPTION OF THE DRAWINGS

FIG. 1 shows a diagram which schematically shows the electrical configuration of the wearable air quality monitor.

FIG. 2A is a flowchart showing the execution of the program for calibrating, sampling, calculating and displaying the CO measurements in the micro-controller.

FIG. 2B is a flowchart showing the execution of the program for sampling, calculating and displaying the PM_{2.5} measurements in the micro-controller.

FIG. 3 is a graph showing the relationship between the detection voltage and the concentration of particulate matter (PM).

FIG. 4 is a graph showing the relationship between the ratio of the carbon monoxide (CO) detector resistance over the clean air resistance and the concentration of carbon monoxide.

FIG. 5A is the 3D CAD model of the wearable air quality monitor that was developed for manufacturing the working prototype shown in FIG. 5B.

FIG. 5B is a picture of an actual working prototype of the unit and the displayed valued of CO and PM.

FIG. 6A is an isometric drawing of the base of the monitor casing showing the dimensions and geometric characteristics.

FIG. 6B is a isometric drawing of the cover of the monitor casing showing the dimensions and geometric characteristics.

FIG. 6C shows the assembly of the mechanical position and fit of the major components of the invention.

### DETAILED DESCRIPTION OF THE INVENTION

Disclosed is a wearable air quality monitor that has a housing with connections that can receive the signals from a CO sensor and a PM sensor. The connections are directly connected to a micro-controller that processes all the functions needed to display the air quality measurements. The monitor may be powered by a battery. The controller performs the heating cycle for the CO sensor and the pulsing cycle for the PM sensor. These cycles allow the optimal performance of the sensors which provides accuracy of the measurements.

The monitor also includes a one-line display that keeps updating based on the sampling and averaging of the data performed by the micro-controller. The system provides for a communication interface allowing communication of the monitor with an external computer.

The disclosed system is unique in being wearable and being able to provide real-time measurements of both CO and PM_{2.5} accurately since the parameters of the solid-state sensors are continuously controlled. In addition, the sensors may be connected and disconnected without any disassembly or the use of any tools. All the components making up the wearable unit are solid state improving the overall reliability. It further has the capability to communicate with an external computer.

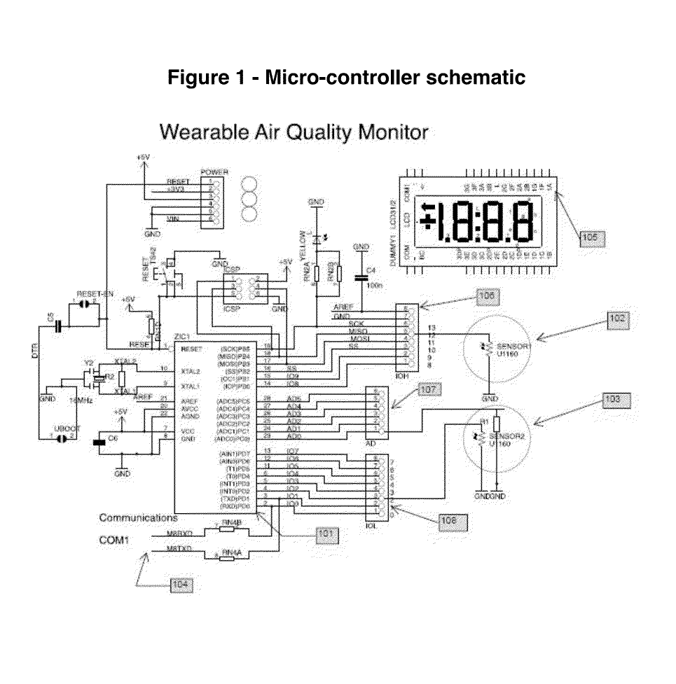

Referring to the drawings more particularly by reference numbers, FIG. 1 shows the micro-controller and circuit board design for the wearable air quality monitor. The system (101) is the micro-controller that performs the sampling, calibration and the cycling of the temperature for the CO sensor and the pulsing signal for the PM sensor. The signals are provided to or from the micro-controller (101) as inputs shown by boxes (106), (107) and (108). The heater signal for the CO sensor (103) is an output from the micro-controller (101) through terminal (107) and is contained within the sensor itself. The sensor resistance (103) is read as a voltage divider input to the controller through terminal (108). The pulsing signal for the PM sensor (102) is another controller (101) output. The voltage from the PM sensor (102) is sent to the controller (101) as an input through terminal (106).

All the sampling and calibration data are performed by the micro-controller (101). The samples are collected as analog signals to the controller through terminal block (107). The data is converted into an 8-bit format that can be processed by the controller. Therefore, the need for air or gas sampling chambers, pumps or air-jets is eliminated.

The system has the the capability to display the measured concentrations of CO and PM_{2.5} through the display (105). Furthermore, the system has communications port allowing communications (104) with an external computer or portable smart device such as a smartphone or a tablet. The port also serves as a means for programming and servicing the controller as well as providing power to all the components in the monitor.

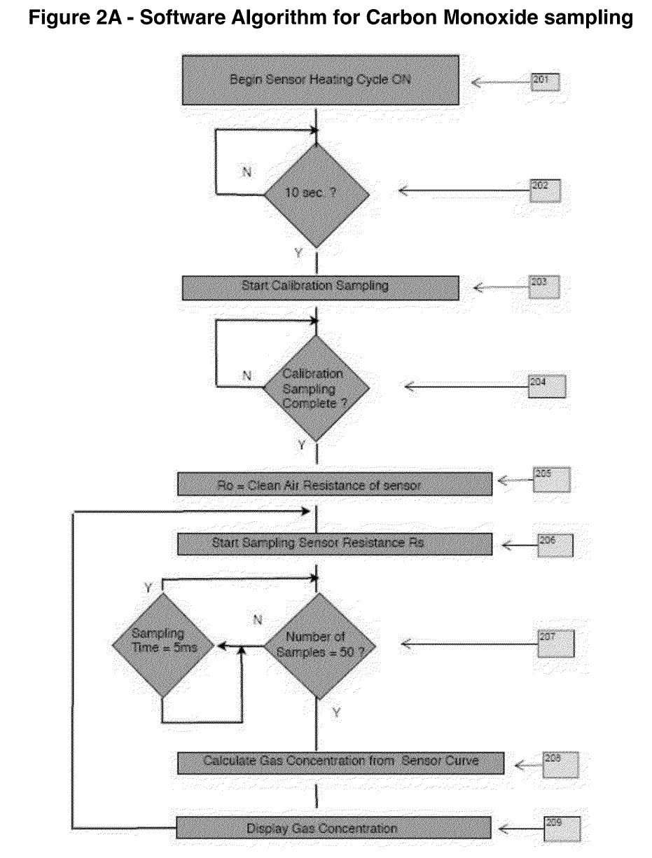

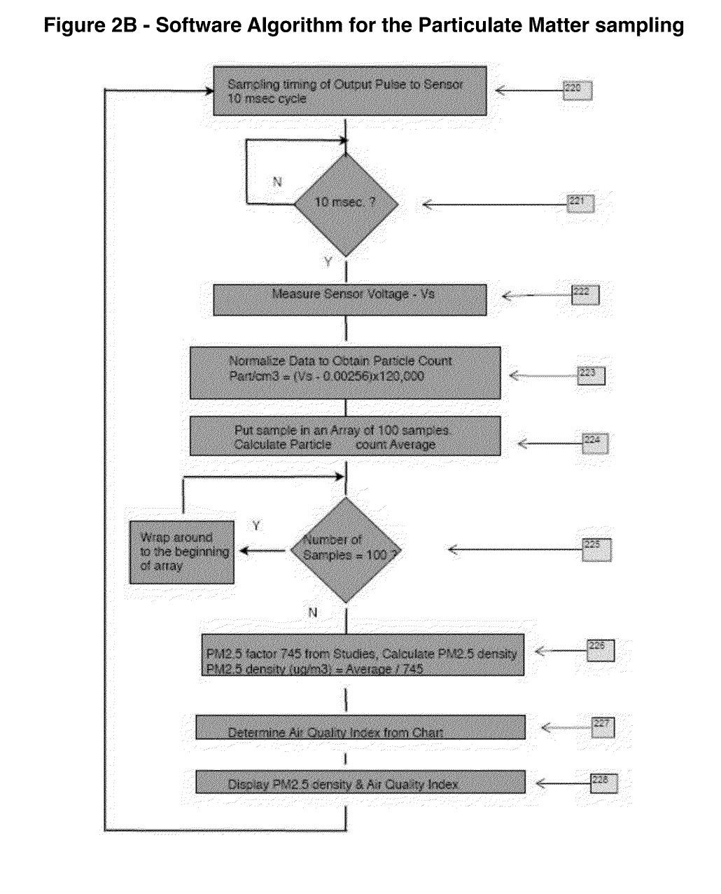

FIG. 2A is a flowchart describing the portion of the micro-controller (101) program that handles the functionality for the CO sensor. As described above, the CO sensor requires clean-air calibration and a controlled sequence for the heating elements to ensure the sensor operates to provide accurate measurements of CO. The program begins by setting the controller (101) output that controls the heater element to ON state (201). The heating element remains in the ON state for 10 seconds (202) to ensure that a proper operating temperature is met. After the initial heating cycle (202), the signal from the CO sensor is sampled by the micro-controller (101) at a rate of 50 samples taken every 50 ms (203). This step should be performed in a clean air area. Calibration is complete once the number of samples has been reached (204). The resistance of the sensor Ro can be measured since the circuit shown in (103) is a voltage divider circuit. This resistance is the clean air resistance Ro (205) that will be needed to measure the CO. Following the calculation of Ro (205), the micro-controller (101) continues sampling in the environment where the CO is to be measured. The sampling is performed in (207), a resistance of the sensor (Rs) is then calculated allowing the micro-controller (101) to calculate the ratio Ro/Rs and determine the concentration of the CO by interpolating (208) on the Ro/Rs vs CO (ppm) curve shown in FIG. 4. The measurement is sent to the display (209).

FIG. 2B is a flowchart describing the portion of the micro-controller (101) program that handles the functionality for the PM sensor. The PM sensor work with an opto-transistor to detect the airborne particles therefore calibration is not required. A timing pulse used for the sensor to determine the intensity of light from the photo-transistor is controlled by the microcontroller (101). The intensity is proportional to the density of particles. The program begins the 10 ms pulsing cycle (220). Once the 10 ms cycle is completed (221), the program measures the voltage from the sensor Vs (222). The sample of the sensor measurement is normalized allowing the sensor to operate with a higher degree of sensitivity in low density and higher density environments. The normalizing function is applied to the sensor measurement Vs in (223). The sample is placed in an array of 100 samples for averaging and smoothing of the data (224). This make the measurement reading more easy to interpret rather than having a discontinuous set of data. The micro-controller (101) maintains the array to ensure that the data is refreshed in a First In, First Out (FIFO) manner (225). At this stage, the measurements are the quantity all particles 0.5 micron and larger per volume of 1 cm³. The PM_{2.5} density is determined by dividing the measurement by a factor of 745. This factor is derived from studies taken in urban and rural settings that accurately quantify the density of PM_{2.5} when measurements of particles are taken per cm³. This calculation is done in (226) giving the density of PM_{2.5} in ug/m³. The Air Quality Index (227) is derived from the EPA/Environment Canada charts that correlate PM_{2.5} density with an Air Quality levels and displayed (228).

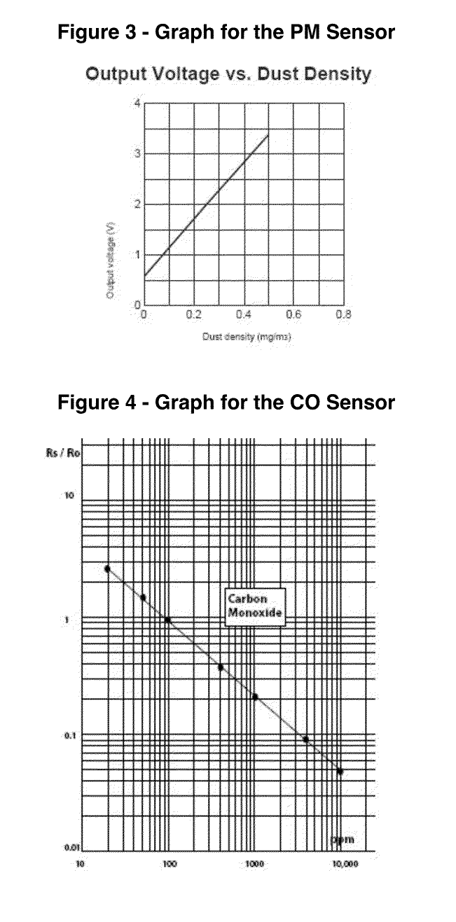

FIGS. 3 and 4 are graphs for the 2 sensors used in the disclosed wearable air quality monitor. They are used by the flowcharts in FIGS. 2A and 2B to translate the sensor signals inputted into the micro-controller (101) into the numeric data needed to perform the measurements describes in the flowcharts. Since the chart in FIG. 4 is on a Logarithmic base 10 scale, the micro-controller (101) must calculate the inverse Log in (208) on FIG. 2A to provide the correct measurement. The linearity of the PM sensor in FIG. 3 is a function type Y=mX+b, where m is the slope and b=0.55 as shown on the graph.

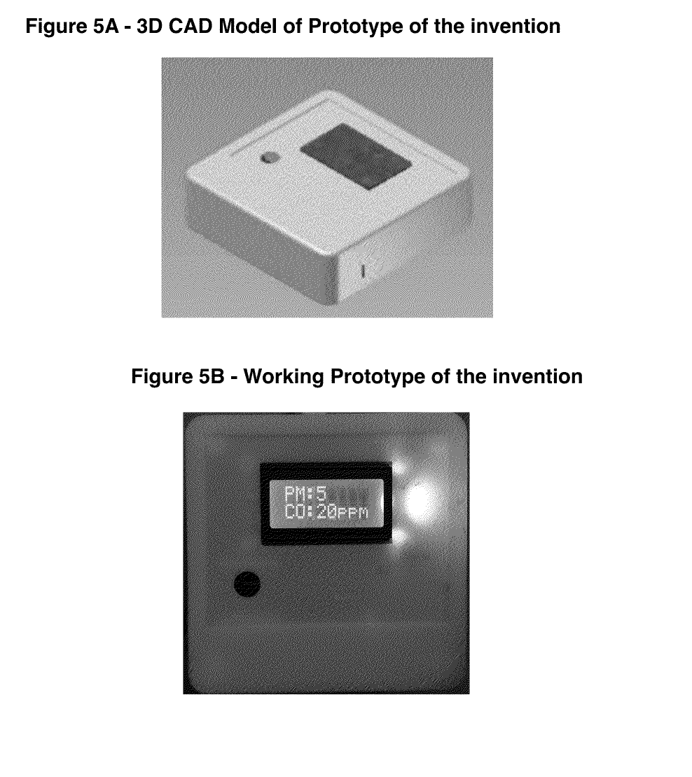

FIG. 5A, 5B, 6A and 6B show an embodiment of a wearable air quality monitor system according to this invention comprising a casing proper made of injection-molded polymer (611). A processing unit, sensors and display are accommodated. The liquid-crystal display panel (607) is fastened in the casing cover so as to be exposed to the outside though an rectangle cutout in the case cover proper. An orifice is provided on one side of the casing proper to accommodate the gas sensor (602). Another orifice on the top of the casing proper is provided to accommodate the particulate matter sensor (603). The gas sensor is fitted inside to the side of the casing by use of a frame (604). The particulate matter sensor is held by a frame molded onto the baseplate proper (601). The two parts of the casing (cover and baseplate) are fastened together by driving four screws through holes provided therein (605).

The design allows for easy change of the sensors when the sensitivity has dropped with prolonged use by unfastening the four screws to the casing baseplate and cover and disconnecting the sensors. The replacement sensors are then reconnected without the use of tools by connecting members pressed into place and fitted into their respective frames.

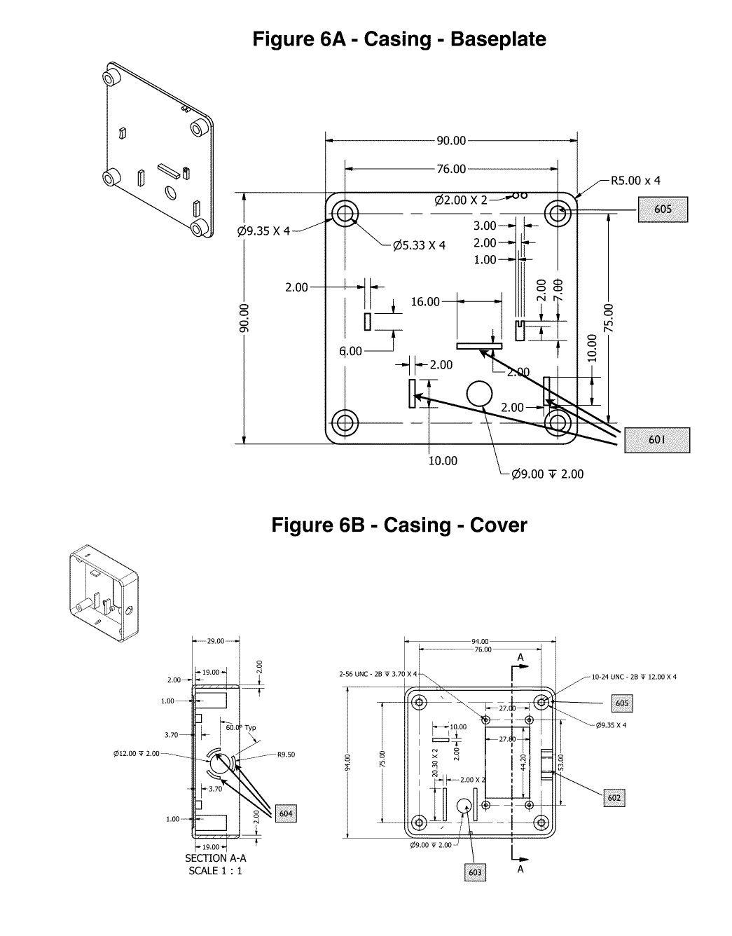

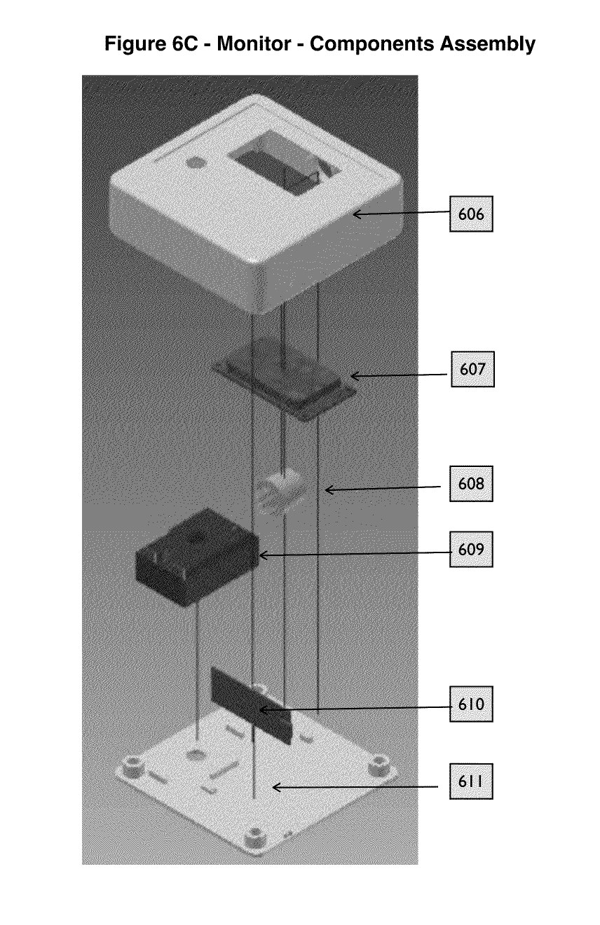

FIG. 6C shows the positions of all the components and how they fit within the whole unit of a wearable air quality monitor system based on the operation described in FIGS. 1-6B. The sensors (608, 609) are pre-wired and connect to the controller of the system (610). The casing, made of two parts (606, 611), house all the design components shown in FIG. 1. The LCD display (607) displays the CO in ppm and the PM_{2.5} in ug/m³. As shown, the monitor is small and contained in a simple case allowing it to be worn and connections are done without the need of tools or disassembly.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various modifications may occur to those ordinarily skilled in the art.

1. A wearable air quality monitor system, comprising:
A housing with a dual-line display that allows for instantaneous measurement and display of a CO and a PM sensor in the unit;

2. The system of claim 1 designed to be worn or carried in a manner similar to a cell phone;

3. The system of claim 1, further comprising a micro-controller that performs the heating cycle for the CO sensor;

4. The system of claim 2, that further performs the auto-calibration of the CO sensor;

5. The system of claim 3 that performs the sampling of the sensor measurements, the interpolation on the CO sensor curve and the calculation to determine the measured ppm of CO;

6. The system of claim 3 that performs the sampling of the sensor measurements, the interpolation on the PM sensor curve and the calculation to determine the measured density of PM;

7. The system of claim 5 that performs the sampling of the sensor and the mathematical calculations to determine the measured density (ug/m3) of PM_{2.5};

8. The system of claim 1, further has communication capability with an external computer, smartphone, tablet.

9. The system of claim 1, further allows the sensors to be connected and disconnected by press connection without the use of tools.

10. A wearable gas monitor comprising a casing having an opening on the side to accommodate gas sensors other than carbon monoxide and an opening on the top to allow for the detection of particulate matter. The gas sensors are attached by polymer clamps embedded on inside of the casing cover.
